(19) United States
(12) Patent Application Publication
(10) Pub. No.: US 2026/0177559 A1
(43) Pub. Date: Jun. 25, 2026

(54) SYSTEMS AND METHODS FOR THE AUTOMATED DIAGNOSIS OF MYCOBACTERIAL INFECTION

(71) Applicant: The Administrators of the Tulane Educational Fund, New Orleans, LA (US)

(72) Inventors: Jia Fan; Tony Hu; Duran Bao; Sudipa Maity

(21) Appl. No.: 19/124,632

(22) PCT Filed: Oct. 26, 2023
(86) PCT No.: PCT/US2023/077966

§ 371 Date: Apr. 25, 2025

Related U.S. Application Data
(60) Provisional application No. 63/419,466, filed on Oct. 26, 2022.
(60) Provisional application No. 63/449,762, filed on Mar. 3, 2023.

(51) Int. Cl. G01N 33/68; C07K 14/47; C07K 14/475

(52) U.S. Cl. CPC G01N 33/6893; C07K 14/4702; C07K 14/4748; C07K 14/475; G01N 33/6848

(57) ABSTRACT

Disclosed herein is a method for diagnosing a mycobacterial infection in a subject. The method comprises performing acetonitrile fractionation of a subject sample to deplete depleted in high molecular weight proteins. The sample is enzymatically digested and analyzed with liquid chromatography tandem mass spectrometry (LC-MS/MS) and bottom-up proteomic analysis to identify a plurality of sample peptides. The sample is then analyzed with a classifier algorithm and a PW species identification score (PWsp) is generated by dividing the number of single and multi-peptide combinations that identify a specific mycobacterium species by the total number of peptide combinations specific for any mycobacteria. A higher PWsp for a given species of mycobacterium indicates a higher likelihood the subject has an infection of that species. This method provides an efficient and accurate way of diagnosing mycobacterial infections in subjects.

### CROSS-REFERENCE TO RELATED APPLICATION(S)

This application claims priority to U.S. Provisional Application No. 63/419,466, filed Oct. 26, 2022, and entitled “Development of Automated Pipeline, PEP-TORCH, for Identifying Mycobacterial Species/subspecies Based on Signature Peptides,” U.S. and Provisional Application No. 63/449,762, filed Mar. 3, 2023, each of which is hereby incorporated by reference in its entirety under 35 U.S.C. § 119(e).

### TECHNICAL FIELD

The disclosure generally relates to a method of detecting and identifying Mycobacterium species, subspecies, and strains, and more particularly relates to a method of identifying Mycobacterium using an automated system based on signature peptides.

### BACKGROUND

The Mycobacterial species is diverse and broadly classified into Mycobacterium Tuberculosis (MTB) and Non-Tuberculosis Mycobacteria (NTM) (e.g., of four commonly and clinically significant Mycobacteria are Mycobacterium Avium (MAV), Mycobacterium abscessus (MAB), Mycobacterium kansasii (MKAN), Mycobacterium intracellulare (MINT)). The symptoms of MTB and NTM may be similar; however, they require different treatments to effect cures. Treating Mycobacteria may be particularly difficult since diagnosis requires long culture and subculture methods ranging from weeks to months. In addition, the current diagnostic method is not efficient in identifying several closely related taxa.

Due to the timely need of therapeutic intervention, the diagnosis of Mycobacterium has greatly evolved from traditional and time consuming biochemical and culture-based methods to faster alternative molecular methods such as Xpert, PCR and MALDI-Biotyper. Although Xpert detects MTB complex, it is less sensitive than sputum culture and does not detect NTM infections. In addition, PCR has been greatly revolutionized to discriminate between MTB and NTM but lacks sensitivity at the sub-species level. For example, PCR cannot further classify M. abscessus as M. massiliense, M. bolletii and M. abscessus subspecies. Only in recent years has PCR been multiplexed and under development to identify between macrolide susceptibility, however, it is time-consuming, relatively expensive, and require experts for the sample preparation and data analysis, which is not ideal for routine clinical use. In addition, traditional PCR or Xpert can only discriminate between MTB complex and NTM and is inefficient in identifying MTB complex subspecies such as M. bovis or M. africanum. Several methods based on sophisticated whole genome sequencing (WGS) can achieve subspecies-level resolution but they are not cost-effective and require expensive equipment. Although rapid identification of NTM with MALDI-TOF MS has outperformed molecular techniques, such as GenoType (Hain Lifescience GmbH, Nehren, Germany), the challenge lies in the large amount of proteins (microbial biomass) needed for the test which limits its implementation early on. Additionally, the Mycobacteria Library database (Bruker Daltonik GmbH, Bremen, Germany) available so far (v2.0) provides low scores, particularly for NTM belonging to the slow-growing groups and currently under development. All the above techniques have restricted usage at the earlier stages of infection and are limited in sub-species level identification which is crucial in case of Mycobacterium.

Therefore, there is the need for a point-of-care test for identifying the species and sub-species of Mycobacterium with high specificity.

### BRIEF SUMMARY

Disclosed herein are methods and systems for diagnosing a Meyobacterial infection in a subject. In one general aspect, method may include performing acetonitrile fractionation of a culture filtrate protein (CFP) sample obtained from the subject and retaining the supernatant fraction, where the retained supernatant fraction is selectively depleted in high molecular weight (HMW) proteins. Method may also include performing an enzymatic digest of the supernatant fraction to produce a digested peptide sample. Method may furthermore include performing liquid chromatography tandem mass spectrometry (LC-MS/MS) and bottom-up proteomic analysis of the digested peptide sample to identify a plurality of sample peptides. Method may in addition include analyzing the plurality of sample peptides with a classifier algorithm, where the classifier algorithm is configured to cross reference each of the plurality of sample peptides with a mycobacterium peptide database to determine which of the plurality of sample peptides, individually or in combination, are specific to a mycobacterium species or subspecies in the database; and generating a PW species identification score (PWsp) by dividing the number of single and multi-peptide combinations that identify a specific mycobacterium species by the total number of peptide combinations specific for any mycobacteria, where a higher (PWsp) for a given species of mycobacterium indicates a higher likelihood the subject has an infection of that species. Other embodiments of this aspect include corresponding computer systems, apparatus, and computer programs recorded on one or more computer storage devices, each configured to perform the actions of the methods.

Implementations may include one or more of the following features. Method where the classifier algorithm further comprises a first section, where the first section is configured to analyze and cross reference each of the plurality of sample peptides to a database and identify all species of organisms for which each of the plurality of sample peptides is associated. Method where the classifier algorithm further comprises an identification section, where the identification section is configured analyze the output for the first section and eliminate all peptides not associated with a mycobacteria species and to output a refined plurality of sample peptides. The method where the classifier algorithm further comprises a scoring section, where the scoring section analyzes the output of the identification section and eliminates all peptides that are not specific to a single mycobacteria species, either alone or combined with a second or third sample peptide and outputting a plurality of species-specific sample peptides. Method where the classifier algorithm is further configured to compile the output of the scoring section and calculate the PWsp score by dividing the number of species specific sample peptides (output of the scoring section) by the number of peptides in the refined plurality of sample peptides (output of the identification section). Method where the classifier algorithm is further configured to repeat the steps for mycobacteria subspecies in order to generate a PW subspecies score (PWsubsp). Method where when two or more species have PWsp score above the predetermined threshold, the subject is diagnosed with a co-infection. Method where the supernatant fraction is enriched in mycobacteria specific proteins. Method where the supernatant fraction is comprised of proteins of about 60 kDa or less. Method where the enzymatic digest is a trypsin digest. Method where the CFP samples are from early-growth mycobacterial growth indicator tube (MGIT) cultures. Method where CFP samples are collected and processed at the first sign of microbial growth. Implementations of the described techniques may include hardware, a method or process, or a computer tangible medium.

In one general aspect, method may include performing LC-MS/MS and bottom-up proteomic analysis on an enzymatically digested HMW depleted CFP sample from the subject and identify a plurality of sample peptides. Method may also include analyzing the plurality of sample peptides with a classifier algorithm, where the classifier algorithm comprises: Method may furthermore include a first section, where the first section is configured to analyze and cross reference each of the plurality of sample peptides to a database and identify all species of organisms for which each of the plurality of sample peptides is associated. Method may in addition include an identification section, where the identification section is configured analyze the output for the first section and eliminate all peptides not associated with a mycobacteria species and to output a refined plurality of sample peptides. Method may moreover include a scoring section, where the scoring section analyzes the output of the identification section and eliminates all peptides that are not specific to a single mycobacteria species, either alone or combined with a second or third sample peptide and outputting a plurality of species specific sample peptides. Method may also include where the classifier algorithm is further configured to compile the output of the scoring section and calculate the PWsp score by dividing the number of species-specific sample peptides (output of the scoring section) by the number of peptides in the refined plurality of sample peptides (output of the identification section). Other embodiments of this aspect include corresponding computer systems, apparatus, and computer programs recorded on one or more computer storage devices, each configured to perform the actions of the methods.

Implementations may include one or more of the following features. Method may include analyzing the plurality of peptides with classifier algorithm to generate a mycobacteria subspecies in order to generate a PW subspecies score (PWsubsp). Implementations of the described techniques may include hardware, a method or process, or a computer tangible medium.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, system may include a module, configured to perform acetonitrile fractionation of a culture filtrate protein (CFP) sample obtained from the subject and retaining the supernatant fraction, where the retained supernatant fraction is selectively depleted in high molecular weight (HMW) proteins and to perform an enzymatic digest of the supernatant fraction to produce a digested peptide sample. System may also include a module configured to perform liquid chromatography tandem mass spectrometry (LC-MS/MS) and bottom-up proteomic analysis of the digested peptide sample to identify a plurality of sample peptides. System may furthermore include one or more processors configured to: analyze the plurality of sample peptides with a classifier algorithm, where the classifier algorithm is configured to cross reference each of the plurality of sample peptides with a mycobacterium peptide database to determine which of the plurality of sample peptides, individually or in combination, are specific to a mycobacterium species or subspecies in the database. System may in addition include generate a PW species identification score (PWsp) by dividing the number of single and multi-peptide combinations that identify a specific mycobacterium species by the total number of peptide combinations specific for any mycobacteria, where a higher (PWsp) for a given species of mycobacterium indicates a higher likelihood the subject has an infection of that species. Other embodiments of this aspect include corresponding computer systems, apparatus, and computer programs recorded on one or more computer storage devices, each configured to perform the actions of the methods.

Implementations may include one or more of the following features. System where the classifier algorithm further comprises a first section, where the first section is configured to analyze and cross reference each of the plurality of sample peptides to a database and identify all species of organisms for which each of the plurality of sample peptides is associated. System where the classifier algorithm further comprises an identification section, where the identification section is configured analyze the output for the first section and eliminate all peptides not associated with a mycobacteria species and to output a refined plurality of sample peptides. System where the classifier algorithm further comprises a scoring section, where the scoring section analyzes the output of the identification section and eliminates all peptides that are not specific to a single mycobacteria species, either alone or combined with a second or third sample peptide and outputting a plurality of species-specific sample peptides. System where the classifier algorithm is further configured to compile the output of the scoring section and calculate the PWsp score by dividing the number of species-specific sample peptides (output of scoring section) by the number of peptides in the refined plurality of sample peptides (output of the identification section). Implementations of the described techniques may include hardware, a method or process, or a computer tangible medium.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

FIG. 1A depicts the sampling of tissue of mycobacterial infected suspects and culturing of the sample in MGIT, according to one implementation.

FIG. 1B depicts the prior art method of growing mycobacterial cultures for analysis.

FIG. 1C depicts one implementation where the mycobacterial culture filtrate is processed to yield a peptidome of low molecular weight peptides.

FIG. 1D depicts the processing of a peptidome through a PEP-TORCH analysis and algorithm to identify the starting mycobacteria infecting the subject, according to one implementation.

FIG. 2A shows SDS-PAGE analysis of 6 samples of mycobacteria peptides before and after precipitation with 50% acetonitrile, according to one implementation.

FIG. 2B shows SDS-PAGE analysis of 6 samples of mycobacteria peptides after acetonitrile precipitation.

FIG. 2C shows a graph of protein concentrations in the soluble fraction of the 6 samples, before and after acetonitrile precipitation, according to one implementation.

FIG. 3A shows a graph of protein concentrations, depicting the relative depletion of the 120 and 66 kDa bands after acetonitrile precipitation.

FIG. 3B shows a graph of protein concentrations, depicting the relative enrichment of the 12 and 40 kDA bands after acetonitrile precipitation.

FIG. 3C shows a graph of the increase of protein number based on molecular weight after acetonitrile precipitation, according to one implementation.

FIG. 3D shows a graph of the increase of peptide number based on molecular weight after acetonitrile precipitation, according to one implementation.

FIG. 3E shows a graph of the peptides identified in the experimental example, with the black line representing the mean value, according to one implementation.

FIG. 4 shows a flow diagram of the entire PEP-TORCH process, according to one implementation.

FIG. 5 shows a sample of algorithm logic as would be coded into the PEP-TORCH algorithm, according to one implementation.

FIG. 6A shows the PEP-TORCH species identification and PW scores for samples identified as Mycobacterium abscessus (Mab) or Mab complex isolates, from the implementation described in the experimental example.

FIG. 6B shows the PEP-TORCH species identification and PW scores for samples identified as mycobacterium kansasii (Mka), from the implementation described in the experimental example.

FIG. 6C shows the PEP-TORCH species identification and PW scores for samples identified as mycobacterium kansasii/Mycobacterium avium co-infection (Mka/Mav), from the implementation described in the experimental example.

FIG. 6D shows the PEP-TORCH species identification and PW scores for samples identified as Mycobacterium avium (Mav), from the implementation described in the experimental example.

FIG. 6E shows the PEP-TORCH species identification and PW scores for samples identified as mycobacterium intracellulare (Min), from the implementation described in the experimental example.

FIG. 7 shows the PEP-TORCH species identification and PW scores for various other mycobacterium strains, from the implementation described in the experimental example.

FIG. 8A shows a collection of graphs ranking species-specific Mtb, Mab, and Mka target peptide by their detection frequency in samples and their total number of peptide combinations, from the implementation described in the experimental example.

FIG. 8B shows a collection of graphs ranking selected Mtb, Mab, and Mka peptide pairs from FIG. 8A and individual Mav and Min peptides based on their higher peak area and −log 10 PSM scores, from the implementation described in the experimental example.

FIG. 8C is a graph showing the detection frequency of the PRM target peptides in CFP samples containing the indicated species, where positive and negative results are indicated by red and blue boxes, respectively, from the implementation described in the experimental example.

FIG. 8D is a pair of graphs showing DDA and PRM peak intensities for the selected MTB target peptides at the indicated time after culture inoculation in culture judged growth positive at day 28 post-inoculation, from the implementation described in the experimental example.

FIG. 9A is a collection of graphs showing the median of −log 10 PSM and −log 10 intensities calculated for each peptide from all samples identified in a species group, from the implementation described in the experimental example.

FIG. 9B is a graph showing 9 MTB-specific peptides with high PSM scores, where the PSM scores were validated by PRM in 14 randomly chosen samples, from the implementation described in the experimental example.

FIG. 10A is a collection of graphs showing peptide sequence differences in Mka and Mtb target peptides, and peptide mapping and homology analysis in targeted peptides of Mav, Min and Mab, from the implementation described in the experimental example.

FIG. 10B shows the sequence differences in Mka and Mtb target peptides and peptide mapping and homology analysis in targeted peptides of Mav, Min and Mab, from the implementation described in the experimental example.

FIG. 10C shows three peptide mapping to two proteins (highlighted in yellow and green) of Mav. The red line indicates the mapped peptide sequences, from the implementation described in the experimental example.

FIG. 11 is a flowchart of an example method 1100, according to certain embodiments.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, “acetonitrile fractionation” refers to the technique for the separation and purification of complex mixtures based on the selective solubility of different compounds in acetonitrile, which is a polar organic solvent commonly used in chromatography. The process involves adding acetonitrile to the mixture, causing the formation of distinct layers based on the solubility of the components. These layers can then be separated to isolate and purify the desired compounds.

Liquid chromatography tandem mass spectrometry (LC-MS/MS) is a powerful analytical technique that is used to separate and identify complex mixtures of molecules in a sample. It involves two separation techniques, liquid chromatography and mass spectrometry, which are coupled together to provide high-performance analysis of samples. In liquid chromatography, the components of a mixture are separated based on their physical and chemical properties by passing through a column containing a stationary phase. The components eluted from the column are then ionized and detected by mass spectrometry. The tandem mass spectrometry part of the technique is particularly useful for identifying and quantifying the different components in a mixture. In this part of the analysis, the ions produced in the first mass spectrometry stage are fragmented and analyzed again in a second mass spectrometry stage. By comparing the fragmentation patterns to reference libraries, allows for the identification of different components in the mixture and determine their relative concentrations. LC-MS/MS is used in a wide range of fields, including environmental monitoring, forensic science, clinical research, and drug discovery, due to its high sensitivity, selectivity, and accuracy.

Bottom-up proteomic analysis refers to a method of identifying and characterizing proteins in a complex biological sample. This technique involves breaking down or digesting proteins into smaller peptide fragments using an enzyme such as trypsin. The resulting peptides are then separated and identified through mass spectrometry analysis and or LC-MS/MS.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is substantially free of particles would either completely lack particles, or so nearly completely lack particles that the effect would be the same as if it completely lacked particles. In other words, a composition that is substantially free of an ingredient or element may still actually contain such item as long as there is no measurable effect thereof.

Disclosed herein is a method for diagnosing a mycobacterial infection in a subject. The method involves obtaining a culture filtrate protein (CFP) sample from the subject and performing acetonitrile fractionation to yield a supernatant fraction. The supernatant fraction is selectively depleted in high molecular weight (HMW) proteins. In certain embodiments, the supernatant fraction is substantially free of proteins larger than about 60 kDa. An enzymatic digest (e.g., a trypsin digest) of the supernatant fraction is then performed to produce a digested peptide sample. Liquid chromatography tandem mass spectrometry (LC-MS/MS) and bottom-up proteomic analysis are then performed on the digested peptide sample to identify a plurality of sample peptides.

Next, a classifier algorithm (also referred to herein as PEP-TORCH) is employed to analyze the plurality of sample peptides. In certain embodiments, the classifier is substantially similar to Pseudocode in Box 1, below. The classifier algorithm is configured to cross reference each of the plurality of sample peptides with a mycobacterium peptide database, which facilitates the determination of which of the plurality of sample peptides individually or in combination are specific to a mycobacterium species or subspecies in the database. After such identification, a PW species identification score (PWsp) is generated by dividing the number of single and multi-peptide combinations that identify a specific mycobacterium species by the total number of peptide combinations specific for any mycobacteria.

A higher PWsp for a given species of mycobacterium indicates a higher likelihood that the subject has an infection of that species. Therefore, the disclosed method provides a reliable and accurate way to diagnose mycobacterial infections in subjects by analyzing the presence of specific peptides in their CFP samples.

According to certain embodiments, the method includes obtaining an enzymatically digested HMW depleted CFP sample from the subject and performing LC-MS/MS and bottom-up proteomic analysis on the sample to identify a plurality of sample peptides (e.g., PEAKS studio search engine, (Taxon ID: 1762)). The identified sample peptides are then analyzed with a classifier algorithm, which includes a first section, an identification section, and a scoring section.

The first section of the classifier algorithm analyzes and cross-references each of the plurality of sample peptides with a database (e.g., through Unipept API code to perform a batch analysis of within the R Studio platform) and identifies all species of organisms for which each of the plurality of sample peptides is associated. The identification section of the classifier algorithm analyzes the output of the first section and eliminates all peptides not associated with a mycobacteria species, resulting in a refined plurality of sample peptides. In certain embodiments, the functions of the first section and identification section are performed by the same section of the classifier algorithm. The scoring section of the classifier algorithm analyzes the output of the identification section and eliminates all peptides that are not specific to a single mycobacteria species alone or in combination with a second or third sample peptide, resulting in a plurality of species-specific sample peptides.

Moreover, the classifier algorithm is further configured to compile the output of the scoring section and calculate the PWsp score. The PWsp score is calculated by dividing the number of species-specific sample peptides (output of the scoring section) by the number of peptides in the refined plurality of sample peptides (output of the identification section). The disclosed method provides an accurate and reliable means of diagnosing mycobacterial infections in a subject using LC-MS/MS and bottom-up proteomic analysis in combination with a novel classifier algorithm.

According to certain embodiments, a PWsp score for a given species above a predetermined threshold indicates positive diagnosis of infection for the species. According to certain implementations, when two or more species have PWsp score above the predetermined threshold, the subject is diagnosed with a co-infection. In certain implementations, any PWsp score above zero indicates infection of a given species.

Further disclosed herein is a sophisticated diagnostic system for identifying mycobacterial infections in a subject. The system comprises several modules: the first module is configured to selectively deplete high molecular weight (HMW) proteins by retaining the supernatant fraction following acetonitrile fractionation of a culture filtrate protein (CFP) sample obtained from the subject, and perform an enzymatic digest of the supernatant fraction to produce a digested peptide sample. The second module is configured for liquid chromatography tandem mass spectrometry (LC-MS/MS) and bottom-up proteomic analysis of the digested peptide sample to identify a plurality of sample peptides. Finally, one or more processors are configured to analyze the plurality of sample peptides with a powerful classifier algorithm that cross-references each of the identified peptides with a mycobacterium peptide database. In certain embodiments, the classifier is substantially similar to Pseudocode in Box 1, below. The classifier algorithm is designed to determine which of the plurality of sample peptides, either individually or in combination, are specific to a mycobacterium species or subspecies in the database. The system is capable of generating a PW species identification score (PWsp) by dividing the number of single and multi-peptide combinations that identify a specific mycobacterium species by the total number of peptide combinations specific for any mycobacteria.

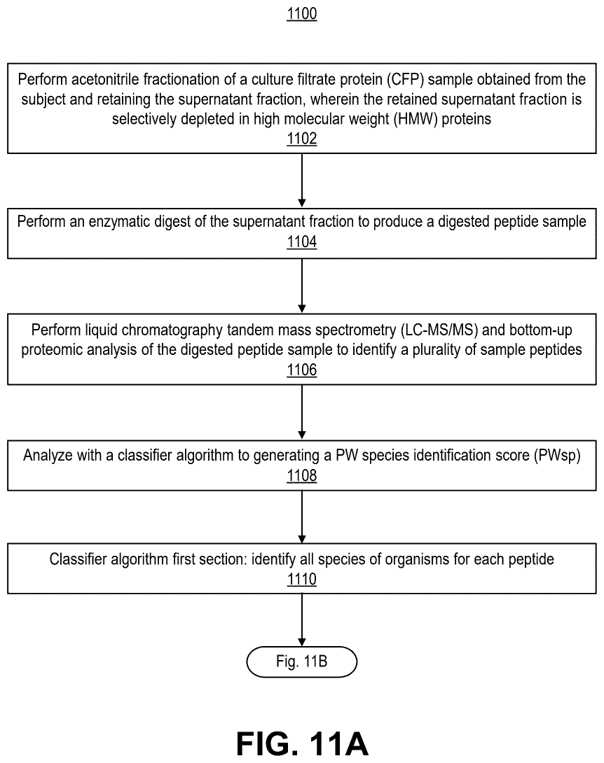

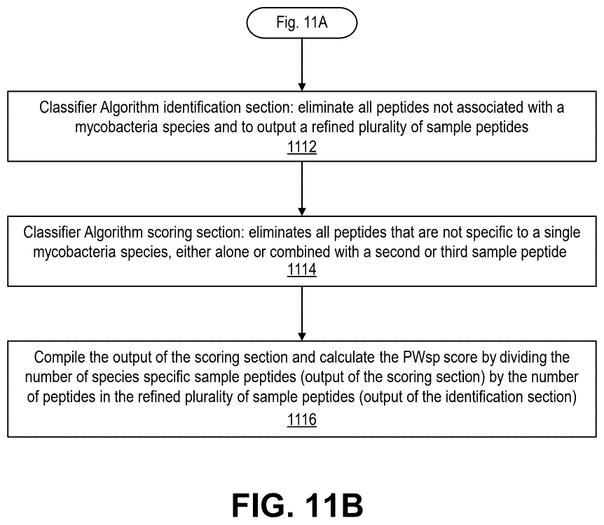

FIG. 11 is a flowchart of an example method 1100.

As shown in FIG. 11, method 1100 may include performing acetonitrile fractionation of a culture filtrate protein (CFP) sample obtained from the subject and retaining the supernatant fraction, where the retained supernatant fraction is selectively depleted in high molecular weight (HMW) proteins (block 1102). For example, a healthcare provider may perform acetonitrile fractionation of a culture filtrate protein (cfp) sample obtained from the subject and retaining the supernatant fraction, where the retained supernatant fraction is selectively depleted in high molecular weight (hmw) proteins, as described above. As also shown in FIG. 11, method 1100 may include performing an enzymatic digest of the supernatant fraction to produce a digested peptide sample (block 1104). For example, a healthcare provider may perform an enzymatic digest of the supernatant fraction to produce a digested peptide sample, as described above. As further shown in FIG. 11, method 1100 may include performing liquid chromatography tandem mass spectrometry (LC-MS/MS) and bottom-up proteomic analysis of the digested peptide sample to identify a plurality of sample peptides (block 1106). For example, a healthcare provider may perform liquid chromatography tandem mass spectrometry (lc-ms/ms) and bottom-up proteomic analysis of the digested peptide sample to identify a plurality of sample peptides, as described above. As also shown in FIG. 11, method 1100 may include analyzing the plurality of sample peptides with a classifier algorithm, where the classifier algorithm is configured to cross reference each of the plurality of sample peptides with a mycobacterium peptide database to determine which of the plurality of sample peptides, individually or in combination, are specific to a mycobacterium species or subspecies in the database; and generating a PW species identification score (PWsp) by dividing the number of single and multi-peptide combinations that identify a specific mycobacterium species by the total number of peptide combinations specific for any mycobacteria, where a higher (PWsp) for a given species of mycobacterium indicates a higher likelihood the subject has an infection of that species (block 1108). For example, a healthcare provider may analyze the plurality of sample peptides with a classifier algorithm, where the classifier algorithm is configured to cross reference each of the plurality of sample peptides with a mycobacterium peptide database to determine which of the plurality of sample peptides, individually or in combination, are specific to a mycobacterium species or subspecies in the database; and generating a pw species identification score (pwsp) by dividing the number of single and multi-peptide combinations that identify a specific mycobacterium species by the total number of peptide combinations specific for any mycobacteria, where a higher (pwsp) for a given species of mycobacterium indicates a higher likelihood the subject has an infection of that species, as described above.

Method 1100 may include additional implementations, such as any single implementation or any combination of implementations described below and/or in connection with one or more other processes described elsewhere herein. In a first implementation, the classifier algorithm further comprises a first section, where the first section is configured to analyze and cross reference each of the plurality of sample peptides to a database and identify all species of organisms for which each of the plurality of sample peptides is associated.

In a second implementation, alone or in combination with the first implementation, the classifier algorithm further comprises an identification section, where the identification section is configured analyze the output for the first section and eliminate all peptides not associated with a mycobacteria species and to output a refined plurality of sample peptides.

In a third implementation, alone or in combination with the first and second implementation, the classifier algorithm further comprises a scoring section, where the scoring section analyzes the output of the identification section and eliminates all peptides that are not specific to a single mycobacteria species, either alone or combined with a second or third sample peptide and outputting a plurality of species-specific sample peptides.

In a fourth implementation, alone or in combination with one or more of the first through third implementations, the classifier algorithm is further configured to compile the output of the scoring section and calculate the PWsp score by dividing the number of species-specific sample peptides (output of the scoring section) by the number of peptides in the refined plurality of sample peptides (output of the identification section).

Although FIG. 11 shows example blocks of method 1100, in some implementations, process 1100 may include additional blocks, fewer blocks, different blocks, or differently arranged blocks than those depicted in FIG. 11. Additionally, or alternatively, two or more of the blocks of process 1100 may be performed in parallel.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Experimental Background

Non-tuberculous mycobacteria (NTM) infections caused by common clinical isolates (e.g., M. avium (Mav), M. intracellulare (Min), M. kansasii (Mka), and M. abscessus (Mab)) produce symptoms similar to tuberculosis (TB) but can require distinct drug regimens so that accurate species or subspecies identifications are crucial for successful management. Mycobacterial culture remains the standard diagnostic approach but can lack specificity and have long latency. Polymerase chain reaction (PCR)-based tests widely used to diagnose TB are less effective for NTM and may not distinguish related taxa. For example, PCR tests used to identify Mab cannot distinguish the Mab subspecies massiliense and abscessus (Mab.subsp.mas and Mab.subsp.abs), these require a different treatment regimens since Mab.subsp.mas is resistant to macrolide drugs. Multiplex PCR, multi-target gene sequencing, and whole genome sequencing (WGS) methods, can have greater specificity but have high turnaround times as they still depend on subsequent solid cultures. Direct WGS of clinical specimens for TB diagnosis has been attempted but has lower sensitivity than WGS of solid cultures. Biotyper instruments that use matrix-assisted laser desorption/ionization time-of-flight mass spectrometry (MALDI-TOF MS) for species identification provide valuable preliminary information to guide treatment options, but often cannot distinguish closely related species, have limited ability to detect drug resistance, and do not detect and identify specific proteins. Biotyper species identifications also rely on matching the spectral profile of a sample against a database of reference spectra obtained from known mycobacteria species, and thus can be influenced by the culture conditions of an analyzed sample and the number of different mycobacterial species isolates available in the reference database.

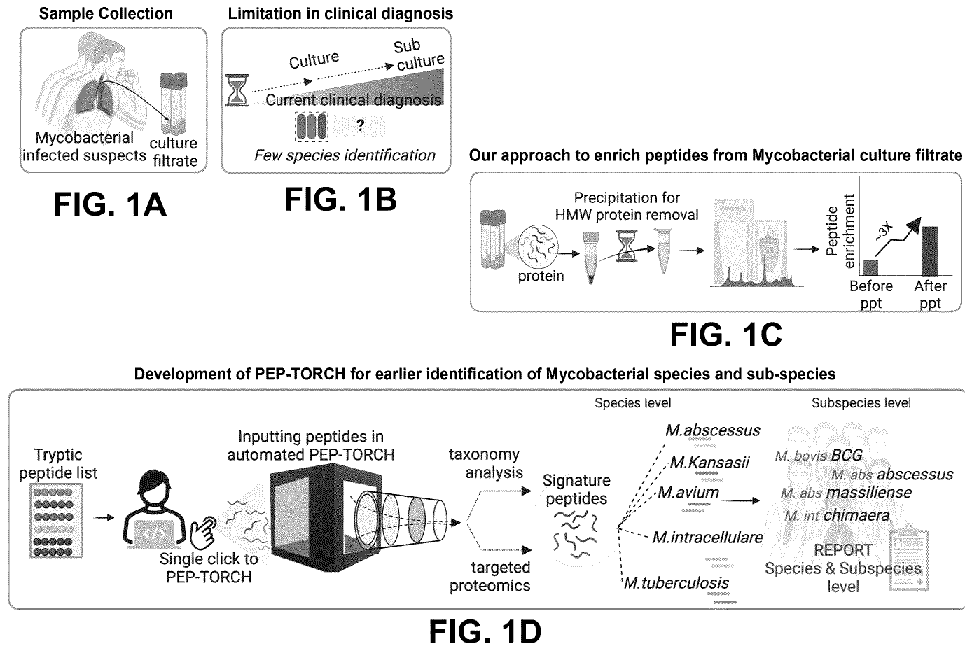

The precise identification of closely related pathogens via peptide sequence variations holds significant potential to improve diagnostic accuracy. LC-MS/MS has the sensitivity and resolution to detect specific sequence variations in peptides that can distinguish closely related taxa within the mycobacterium superfamily to permit specific diagnoses. However, translation of LC-MS/MS diagnostic applications into clinical laboratories has been hampered by the complexity of their sample preparation and data analysis methods. To address the limitation of diagnosis of mycobacteria by long sub-culture methods, a streamlined method was developed to process culture filtrate protein (CFP) samples of early-growth mycobacterial growth indicator tube (MGIT) cultures for LC-MS/MS analysis, see FIGS. 1A-C, and an automated Peptide Taxonomy/Organism Checking (PEP-TORCH) pipeline approach to identify species/subspecies-specific mycobacterial peptide signatures, see FIG. 1D. This PEP-TORCH methodology expedites the diagnostic timeline and uses a unified workflow that confers distinct advantages over the intricate, multi-step, time-intensive clinical approaches currently used to identify specific mycobacterial infections by using peptide sequence information from an extensive public database and a decision tree algorithm to identify distinctive peptide patterns specific for distinct mycobacteria species, subspecies, and strains. All clinical samples analyzed in this study were part of a validation study employed to evaluate the diagnostic accuracy of the PEP-TORCH algorithm. This study encompassed a cohort of 73 clinical samples which represents a notable proportion of the annual United States NTM cases.

### Example Step 1—Acetonitrile Fractionation of CFP Samples Improves Mycobacteria Proteome Coverage

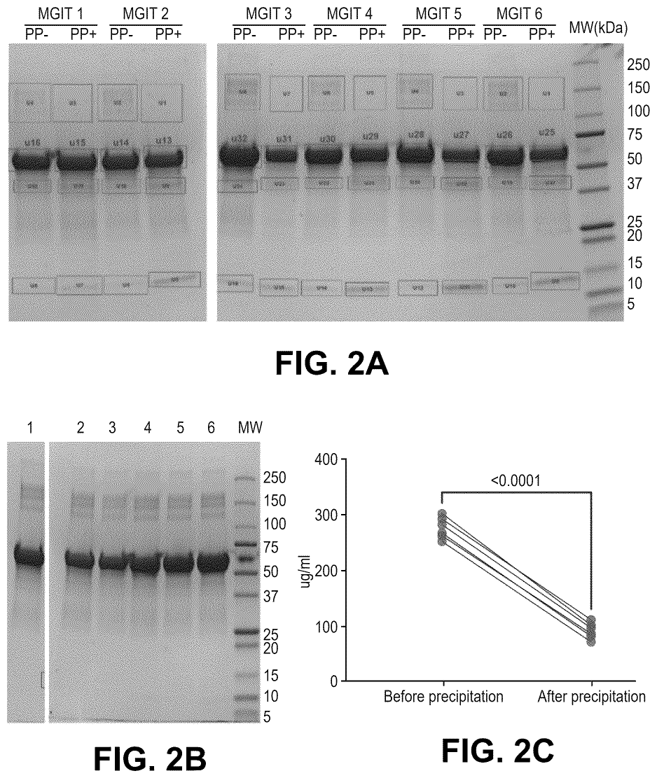

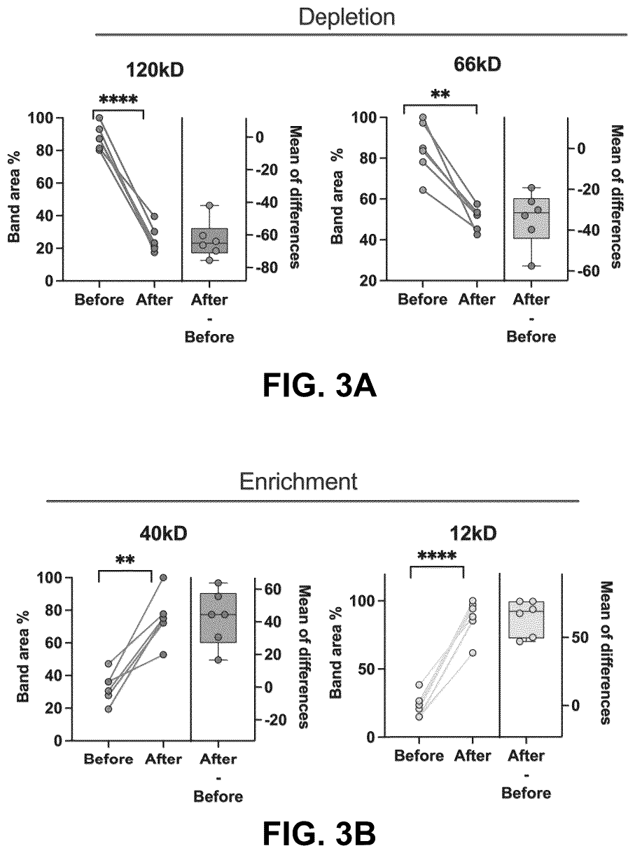

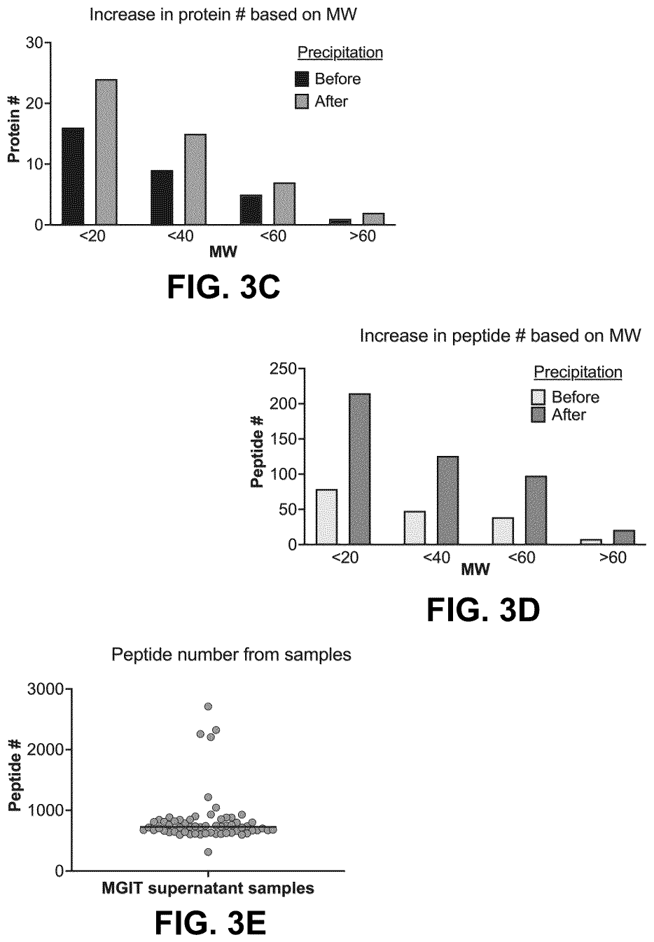

CFP samples isolated from mycobacteria growth indicator tube (MGIT) cultures of 62 clinical specimens at the first sign of microbial growth were subjected to an acetonitrile precipitation procedure to selectively deplete abundant high molecular weight (MW) proteins that could suppress mass spectrometer (MS) detection of less abundant and lower MW mycobacteria-derived proteins. This procedure depleted ˜70% of all MGIT CFP protein (71.3±2.4%) as measured by Bicinchoninic acid (BCA) assay. This can be seen in FIGS. 2A, 2B, and 2C. FIG. 2A shows Sodium dodecyl-sulfate polyacrylamide gel electrophoresis (SDS-PAGE) analysis of protein size distributions in the CFP samples of six growth-positive MGIT CFP supernatant before (PP−) and after (PP+) precipitation with 50% acetonitrile as analyzed in gels stained with Coomassie blue. Boxed regions indicate the regions analyzed to determine protein band areas depicted in FIGS. 3A-3E. FIG. 2B shows SDS-PAGE analysis of size distribution of proteins in the acetonitrile-precipitated fractions of six MGIT samples analyzed in FIG. 2A. FIG. 2C shows protein concentrations of the soluble fraction of these six CFP samples before and after acetonitrile precipitation.

SDS-PAGE analysis of six randomly selected CFP samples detected substantial decrease in 120 kilodaltons (kDa) and 66 kDa bands possibly corresponding to major protein components of the MGIT media, shown in FIG. 3A, and a marked increase in two prominent 40 kDa and 12 kDa protein bands, shown in FIG. 3B. FIG. 3A shows the relative depletion of 120 and 66 kDa protein bands from media. FIG. 3B shows the relative enrichment 12 and 40 kDa mycobacteria-derived proteins bands after acetonitrile precipitation of six CFP samples, depicting individual band areas converted to a percentage scale with the largest band area representing 100. The corresponding box and whisker plot represents the differences in the band widths for each sample in the box and the whiskers at the highest, mean and the lowest band width difference. Two-tailed statistical analyses were performed in both the depletion and enrichment analysis. ****p<0.0001 and **p<0.001.

Upon liquid chromatography with tandem mass spectrometry (LC-MS/MS) analysis, it was found that peptides retained in the supernatant after acetonitrile precipitation of these six sample pairs preferentially mapped to mycobacterial proteins <60 kDa and that these samples identified more peptides and proteins overall (162%±8 and 64%±26, respectively), shown in FIGS. 3C and 3D. FIG. 3C shows the mean number of peptides mapping to protein indicated size ranges. FIG. 3D shows identified proteins in these size ranges as determined using MS data generated before and after acetonitrile precipitation for these six samples. Both protein and peptide # change were significant with p=0.002 and 0.0005 respectively.

Subsequent analysis of the LC-MS/MS spectra of all 62 trypsin-digested CFP samples by bottom-up proteomics and database searches (Mycobacteriaceae, taxonomy ID: 1762) identified similar numbers of peptides (729±125) per sample, shown in FIG. 3E. FIG. 3E shows the number of peptides identified by in these samples belonging to each indicated species or complex, with their mean values represented by the black line for each group.

### Example Step 2—the PEP-TORCH Algorithm Classifies Mycobacteria Species and Subspecies

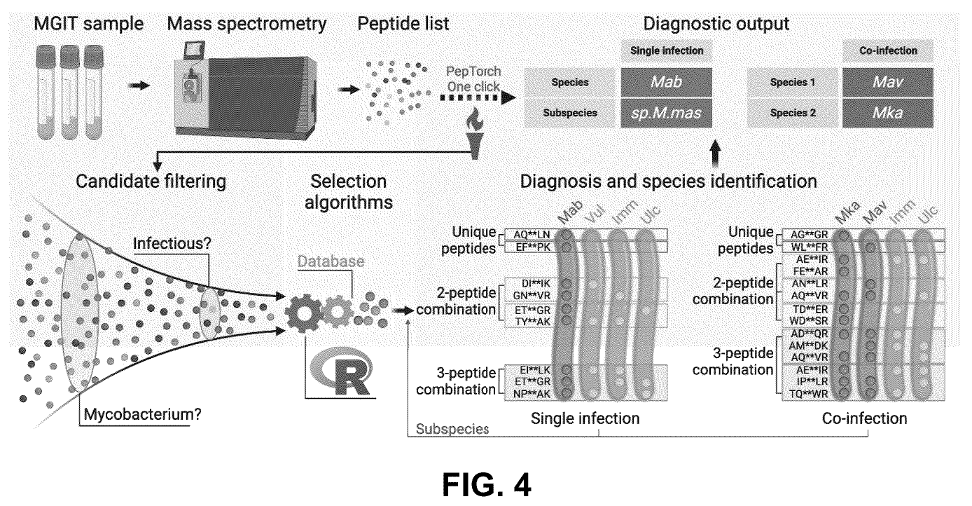

LC-MS/MS data can detect minor sequence variations in peptides from homologous proteins of distinct species or subspecies, which can distinguish distinct mycobacterial isolates. However, this approach requires expertise with both MS and bioinformatics data analyses. To address these issues, an automated PEP-TORCH algorithm was developed that uses a decision tree pipeline to scan peptidomes to produce MS peptide lists that can be used to identify distinct species and subspecies using a unique peptide-weighted (PW) scoring system. This is shown in FIG. 3, specifically, the upper portion, labeled A. FIG. 3 show in portion A, the tryptic peptide list from MGIT sample analysis by MS were used by automated PEP-TORCH as input to give a direct output of species and sub-species in the sample along with their peptide weightage scores. FIG. 4 shows in portion B, the filtering criteria used to slim down the peptide taxon matrix is based on peptides found in Mycobacteria and if infectious to humans. The decision tree in the algorithm used for taxon identification relies on finding unique peptides and two and three peptides unique when combined provided the species and subspecies identification.

Most search engines do not permit batch queries and lack the granularity required to restrict analyses to mycobacteria proteomes derived from clinical samples and identify peptides and peptide combinations specific for pathogenic mycobacteria species, subspecies, and strains. LC-MS/MS data from each clinical CFP sample was therefore analyzed against mycobacterial database entries (Taxon ID: 1762) with the PEAKS studio search engine to produce a list of identified peptides.

The first section in this algorithm used Unipept application programming interface (API) code to perform a batch analysis of all peptides identified from a clinical MGIT sample within the R Studio platform. The next node eliminated all peptide database matches lacking annotations indicating they were derived from mycobacteria or a human isolate origin. Remaining matches were then categorized by their ability to identify a single species, either alone or paired with a second or third peptide, and those that could not do so were excluded from further analyses. This is shown in FIG. 4, specifically, the lower portion, labeled B. Searches performed to identify multi-peptide biomarker signatures were limited to three-peptide combinations as the increased computational workload required for these analyses did not yield significantly improved species or subspecies identifications.

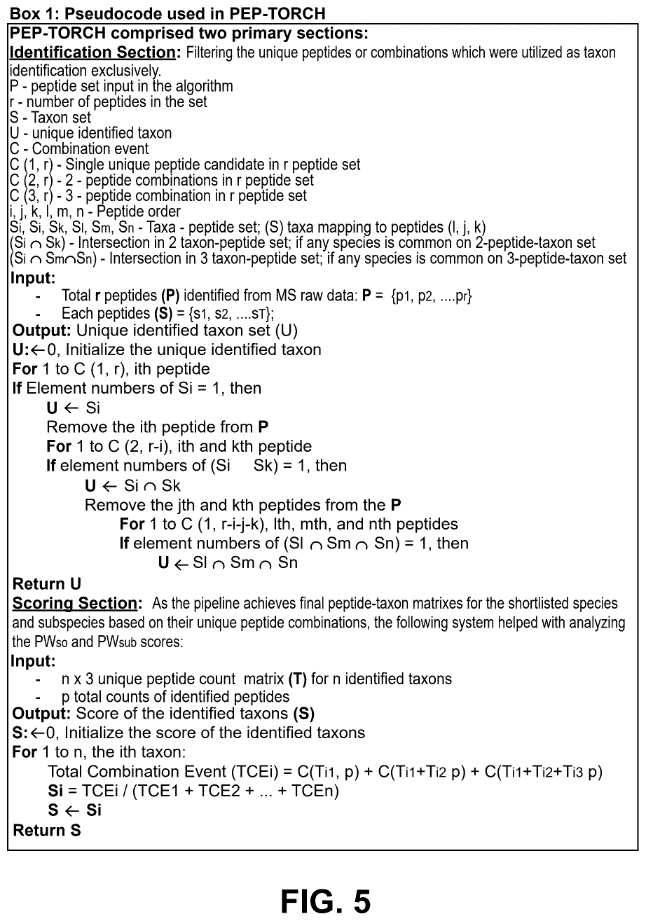

All species-specific peptides and peptide combinations for a sample were then compiled and used to calculate a PW species identification (PWₛₚ) score, calculated by dividing the number of single and multi-peptide combinations that identify a specific mycobacterium by the total number of peptide combinations specific for any mycobacteria. An example of code used to accomplish this calculation can be seen in FIG. 5. Such PWₛₚ scores were primarily determined by multi-peptide combinations since small numbers of peptides could form numerous permutations to generate extensive arrays of multi-peptide combinations. Sporadic detection of single peptide matches to discordant mycobacteria was therefore unlikely to decrease consensus PWₛₚ scores, reduce diagnostic confidence, and alter the number of correct and incorrect identifications. Secondary analyses used to identify subspecies-specific single- or multi-peptide biomarkers and to calculate PW subspecies (PW_{subsp}) scores were determined in the same manner.

### Example Step 3—PEP-TORCH's Automated Mycobacteria IDs Match Those from Multiple Clinical Assays

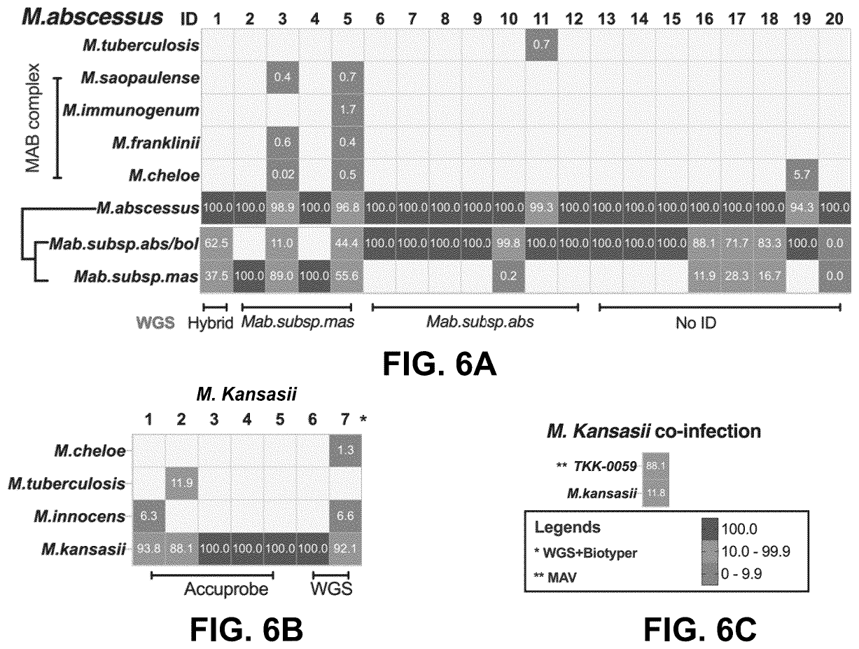

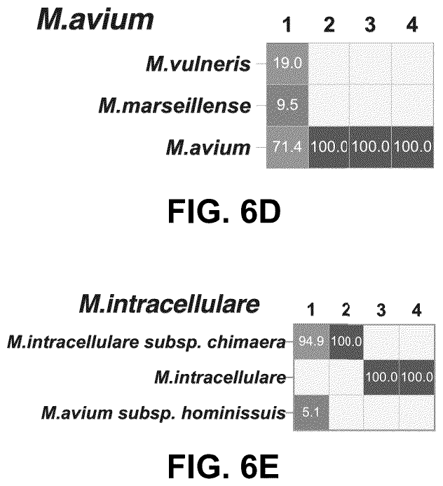

PEP-TORCH classification performance was assessed using CFP from 62 mycobacteria isolates previously evaluated by MALDI Biotyper, Accuprobe, and/or Whole Genome Sequencing (WGS), and its results demonstrated complete agreement with species-level identifications provided by these slower and more labor-intensive methods. The results are summarized in FIG. 6A. FIG. 6A shows PEP-TORCH species identifications and PW scores for samples identified as MAB or MAB complex isolates by MALDI Biotyper results (top), and their PW score for classification as a macrolide-resistant or sensitive MAB subspecies and WGS identification, as available (bottom).

PEP-TORCH's outcomes further provided subspecies identifications for 38 samples, predominantly among Mtb and Mab isolates. Notably, the results were in 100% agreement (17 out of 17) with clinical WGS results for the 17 samples that were classified using both methods. These findings highlight PEP-TORCH's superiority in timely and comprehensive pathogen identification.

All CFP samples identified as Mab-positive by clinical results were judged Mab-positive by PEP-TORCH results, and most (75%; 15/20) had PWₛₚ values that reflected peptide matches only with Mab (Mab PWₛₚ=100). The remaining CFP samples also had high Mab PWₛₚ scores (94.3 to 99.8; 97.8±2.2) that were slightly reduced by sporadic peptide matches with one or more Mab complex species, which had weak PWₛₚ scores (0.4 to 5.7). This data is shown in FIG. 6B. FIG. 6B shows PEP-TORCH PW scores and MALDI Biotyper results for samples identified as Mka.

Peptide matches to non-Mab species likely reflected artifacts rather than mycobacteria co-isolates or contaminants since all of these matched slow growing mycobacteria that would be at a major growth disadvantage versus fast growing Mab complex species, and several matched species rarely detected in human samples. Eliminating peptides associated with rarely detected clinical isolates did not substantially affect the Mab PWₛₚ scores of these samples (mean 97.77 vs. 97.85), since none of sporadic peptide matches produced PWₛₚ scores>0.16 and no samples matched>3 rare isolates.

Robust differential Mab PW_{subsp} scores (>80) were detected in most identified Mab samples to distinguish potential Mab.subsp.mas (5 cultures; 93.1±8.3), Mab.subsp.abs (9 cultures; 93.2±8.0), and M. abscessus subsp. boletii (Mab.subsp.bol) (2 cultures PW_{sub} score: 100±0) samples. Three additional Mab samples had only modest plurality or majority PW_{subsp} scores (52.5±12.1) for Mab.subsp.abs, and the MAB20 isolate did not have an PW_{subsp} score as all identified peptides were shared among these MAB subspecies.

Since Mab subspecies identifications need only distinguish macrolide-sensitive (Mab.subsp.mas) and macrolide-resistant (Mab.subsp.abs or Mab.subsp.bol) isolates to have clinical utility, the ability of PEP-TORCH results to distinguish Mab cultures belonging to these two categories was evaluated. Most samples (85%; 17/20) produced identifications with high overall PW_{subsp} scores (92±16), and most samples (63%; 12 of 19) with subspecies data had perfect PW_{subsp} scores. WGS data indicated MAB1 as a “hybrid species” isolate since it had perfect matches with the Mab.subsp.abs hsp65 and secA genes and the Mab.subsp.mas rpoB gene. This data perfectly matched with PEP-TORCH algorithm with scores of 62.5 and 37.5 for Mab.subsp.abs and Mab.subsp.mas respectively. In addition, PEP-TORCH also classified MAB 3 and MAB5 as hybrid based on the true peptide identifications for both the subspecies, however, for these two samples WGS could recognize only Mab.subsp.mas.

Samples identified as Mka-positive by the pipeline had a mean Mka PWₛₚ score of 82.6±12.1% that increased to 96.3±4.9% after eliminating residual values of rare isolates other than the Mka complex species M. innocens. This data is shown in FIG. 6C. Clinical identification of these samples relied on multifaceted approach, including a combination of Biotyper, Accuprobe and WGS data. FIG. 6C shows PEP-TORCH PW scores and MALDI Biotyper results for samples identified as MkalMav.

A sample classified as a co-infected Mka/Mav sample (co-inf 1) identified by Biotyper results had PWₛₚ scores of 88.1 and 11.9 for Mka and M. TKK-01-0059, respectively. This is shown in FIG. 6D, which shows PEP-TORCH PW scores and MALDI Biotyper results for samples identified as Mav.

However, at least one source classifies M. TKK-01-0059 as belonging to the M. avium complex, while the National Centre for Biotechnology Information (NCBI) classifies it with the Mtb complex. Other samples PEP-TORCH identified as Mav-positive had variable consensus Mav PWₛₚ scores (46.2 to 100; mean 78.2±26.5), and two had matches with ≥2 species. However, after excluding matches to rare clinical isolates, their mean Mav PWₛₚ scores increased (92.9±14.3), with three revealing perfect Mav matches. FIG. 6E shows the PEP-TORCH PW scores and MALDI Biotyper results for samples identified as Min.

Four other samples were judged Min-positive by their strong consensus Min PWₛₚ scores (98.7±2.5), with two further identified as Min subsp. chimera isolates. Few species-specific peptides were detected in three of these four Min-positive samples, including both those without subspecies identifications, but this still represented an improvement over current clinical matrix-assisted laser desorption/ionization-time of flight (MALDI-TOF) results, which cannot distinguish Min and M. chimaera isolates.

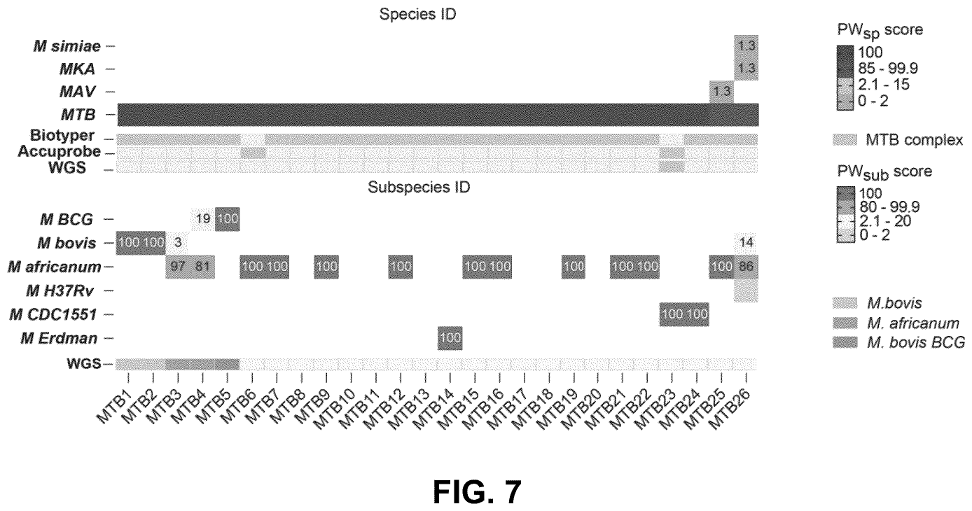

Finally, all 26 samples identified as MTB complex by Biotyper or Accuprobe results had Mtb PWₛₚ scores>97%, most (24 of 26) with 100% scores. Scores are compiled in FIG. 7, which shows MGIT samples classified as MTB or MTB complex species such as M. bovis and M. africanum by PEP-TORCH were otherwise classified only at the MTB complex level by a combination of current clinical methods. The PWsp matched to a 100 for 24 of 26 (˜92%) samples with at least a PWsp of 97 across all samples. PEP-TORCH showed high agreement with limited WGS-validated samples, correctly identifying M. bovis with a 100% match and M. africanum with 81-91% match.

PEP-TORCH results also provided subspecies identifications for 19 of these samples. However, since the Unipept database identifies M. bovis and M. africanum as “sub-variants of Mtb” and thus uses a subspecies rather than species taxonomy when classifying them, these species were identified as Mtb subspecies in the PEP-TORCH results. This analysis provided putative identifications for 13 M. africanum and 3 M. bovis species and 2 Mtb strain isolates (Erdman and CDC1551) with high overall PW_{subsp} scores (98.0±5.4) and the majority (84%) bad perfect PW_{subsp} scores. Notably, there was complete concordance between WGS and PEP-TORCH identifications. Further, only 7 of the Mtb samples did not yield distinct Mtb subspecies/strain identifications, and this was due to peptides sequence conservations among strains such as CDC1551, H37Rv and H37Ra.

### Example Step 4—PEP-TORCH Results Permit Species Identification from Early-Growth Cultures

Parallel reaction monitoring (PRM) was next used to evaluate the biomarker utility of two to three peptide signatures identified by PEP-TORCH for each of the five target species when they were analyzed by a low-resolution triple quadrupole MS system suitable for use in clinical applications. These peptide targets were chosen based on two criteria.

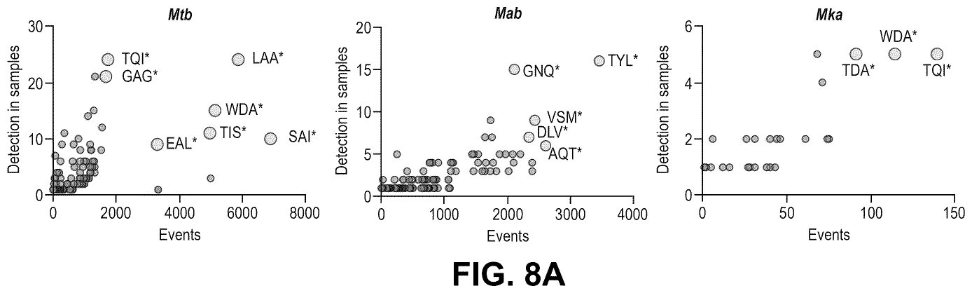

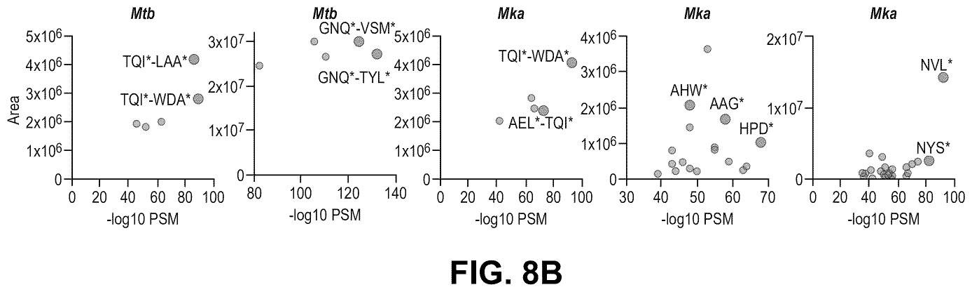

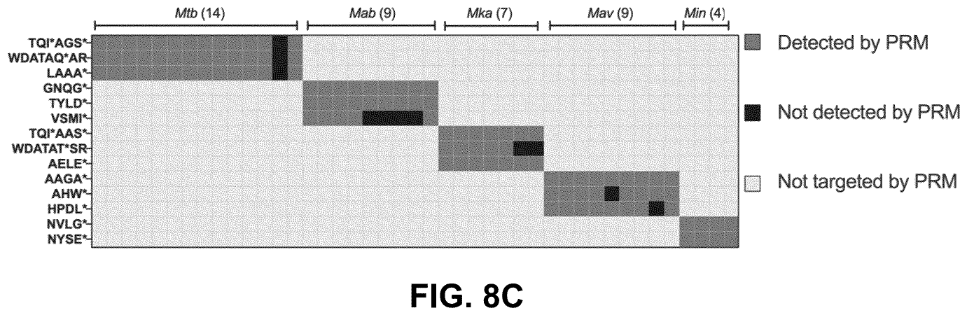

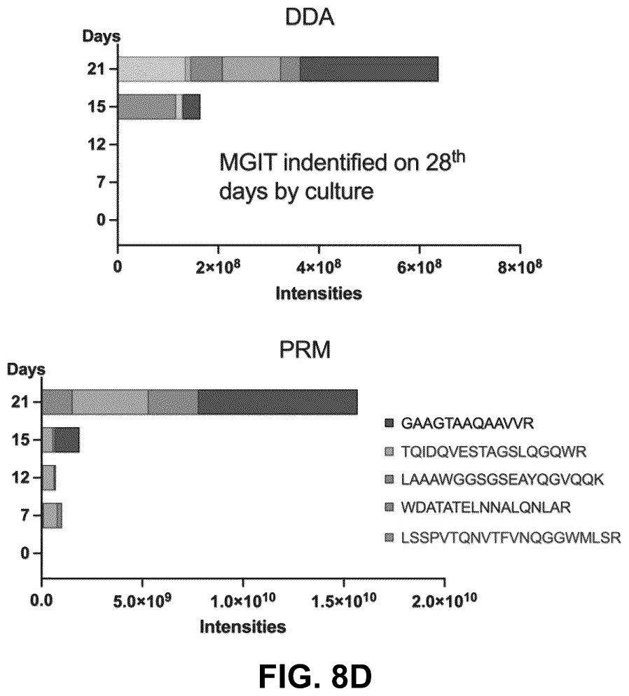

First, peptides were selected by their detection frequency in all samples judged positive for a given species or complex by PEP-TORCH and clinical data, and by their relative frequency in peptide combinations specific for these groups. This data is shown in FIG. 8A, which gives the Ranking of species-specific MTB, MAB, and MKA target peptide by their detection frequency in samples and their total number of peptide combinations to select those with high detection×combination product scores.

This was done to select peptides with robust species-specific expression and sufficient combinations to produce robust PW scores. This approach identified six Mab, four Mka, and seven Mtb peptides that contributed to the five most frequent peptide combinations for each species. Mav and Min peptides were not included in this analysis since they derived from relatively few samples, which lacked species-level identifications by other methods and yielded lower numbers of species-specific peptides than the Mab, Mka, and Mtb samples. Mav and Min peptides selected for further analysis were therefore instead chosen by their ranked detection frequency in the list of unique peptides specific for each of these species.

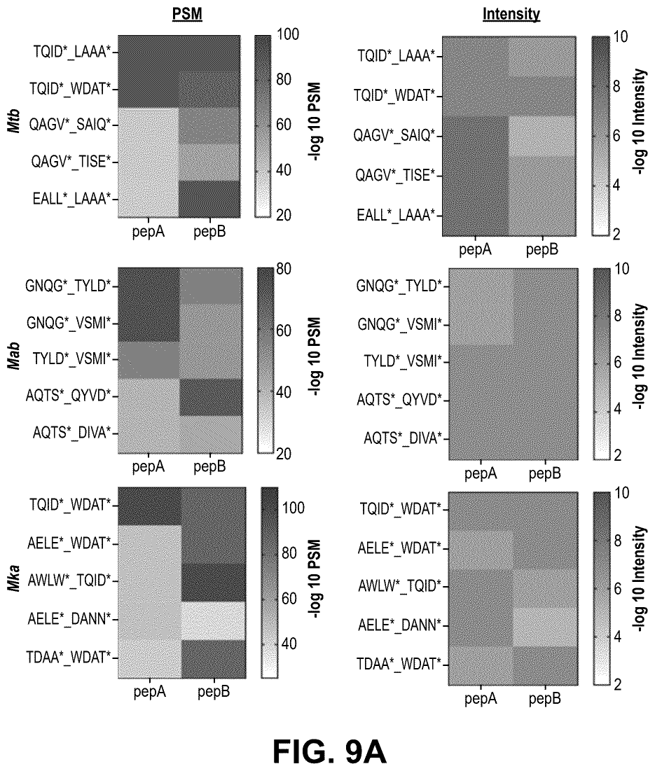

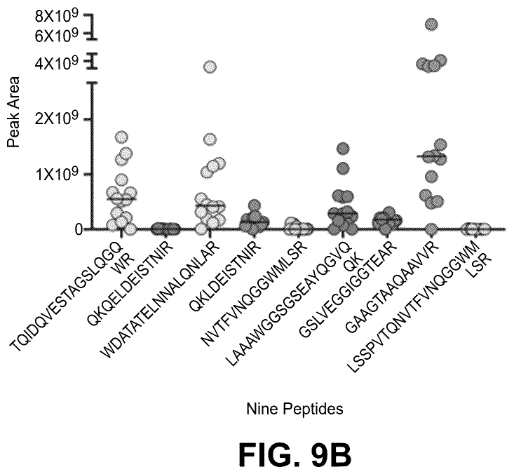

Next, Mab, Mka, and Mtb peptides that met these criteria were screened to identify peptide combinations that produce the highest median DDA peak area values and peptide-spectrum match (PSM) scores to identify peptide pairs that had strong MS signal intensities and confident database identifications expected to allow their consistent detection in MGIT samples. This data is in FIG. 9A, which gives the median of −log 10 PSM and intensities were calculated for each peptide (PepA and pepB in a combination) from all the samples that were identified in a species group. This way the top five peptide combinations were identified based on the highest-log 10 PSM and log 10 intensity values in each species group. From these combinations, three peptides were selected for further PRM targeting for each species group.

This analysis identified 3 peptides that produced peptide pairs specific for Mtb, Mab, and Mka and had the highest average combined scores in samples for each of these species, shown in FIG. 8B. FIG. 8B gives the Ranking of selected MTB, MAB, and MKA peptide pairs from (A) and individual MAV and MIN peptides based on their higher peak area and −log 10 PSM scores. Selected peptides or peptide pairs are indicated by colored data points for each species.

In addition to 3 peptides in Mtb samples, six more peptides detected by DDA that were unique to Mtb and had high PSM scores (−log₁₀ p>55), were also tested by PRM in 14 randomly chosen Mtb-positive MGIT cultures, see FIG. 9B. FIG. 9B shows all nine MTB-specific peptides with high PSM scores (−log₁₀ p>55), that were validated by PRM in randomly chosen 14 samples. The blank line represents the mean peak area of the peptides which corresponds to 10⁶-10⁸ in average. Mav and Min peptides were analyzed in a similar manner to identify single peptides that had the highest combined DDA peak area values and PSM scores, and this approach identified 3 Mav- and 2 Min-specific target peptides.

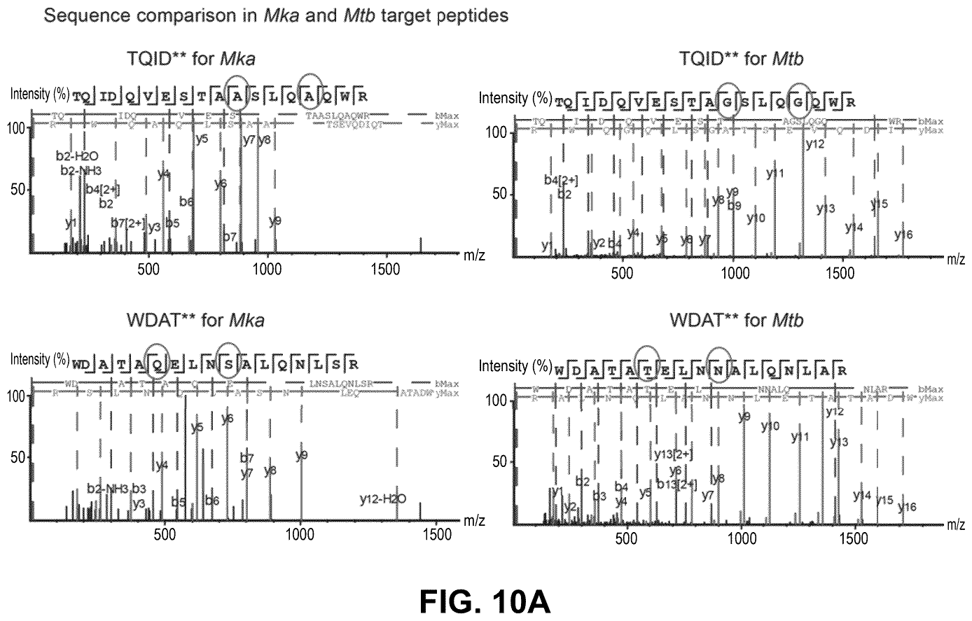

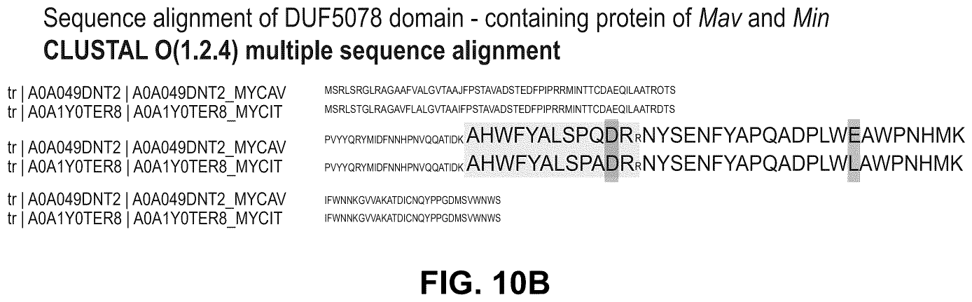

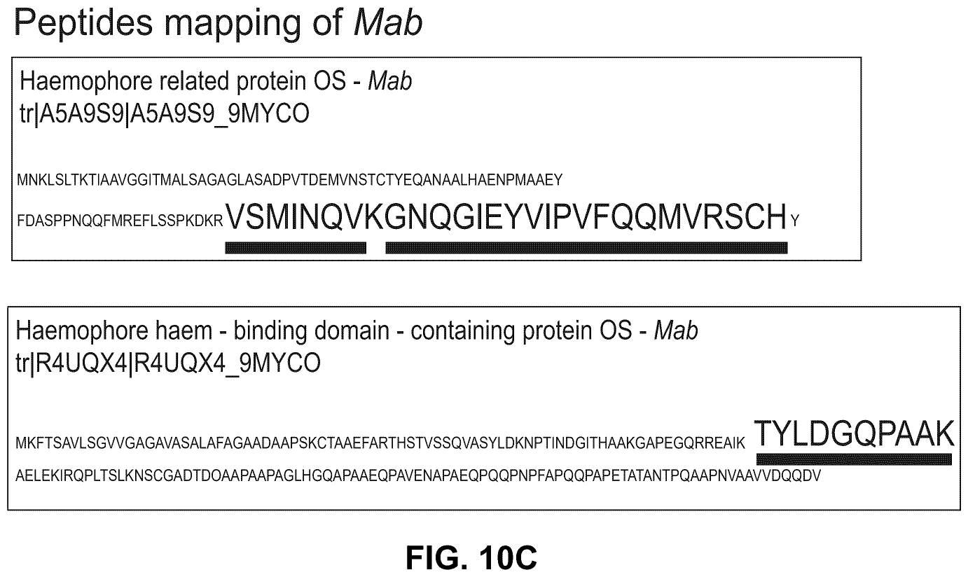

The three selected MTB peptides derived from two secreted factors that play important roles in virulence: ESAT-6 (LAAAWGGSGSEAYQGVQQK and WDATATELNNALONLAR) and CFP-10 (TQIDQVESTAGSLQGQWR). Similarly, the Mka peptides mapped to the Mka orthologs of ESAT-6 (WDATAQELNNALQNLSR) and CFP-10 (TQIDQVESTAASLQAQWR and AELEEISTNIR), although peptides that mapped to the same sites in these proteins differed at two positions, see FIG. 10A. FIG. 10A shows that Mka and Mtb target peptides mapping to the same sites in their respective ESAT-6 and CFP-10 proteins differed at two residues to allow their robust discrimination upon MS analysis of CFP samples of these two species. Mab, Mav, and Min do not express these two virulence factors. Two Min (NYSENFYAPQADPLWLAWPNHMK) and Mav (AHWFYALSPQDR) peptides instead mapped to a protein that is highly conserved between these species (95.9% identity) but differs at single positions in these two matching Mav and Min peptides, see FIG. 10B. The two remaining Mav peptides (HPDLHQQLQQR and AHWFYALSPQDR) mapped to a putative thiol peroxidase and a heme-binding protein, while the two additional Min peptides (NYSENFYAPQADPLWLAWPNHMK and NVLGHLTAANADVNALYQWWR) matched a putative serine/threonine kinase. Finally, all three Mab peptides (VSMINQVK, GNQGIEYVIPVFQQMVR and TYLDGQPAAK) mapped to a hemophore-related protein.

These PRM peptide targets were used to identify mycobacteria species present in all the MGIT samples with sufficient material remaining for a second LC-MS/MS analysis. Fourteen MTB complex samples had sufficient material for this analysis, but few Mab, Mka and Mav samples had sufficient material and new samples were therefore added to these groups, see FIG. 8C. FIG. 8C shows the detection frequency of the PRM target peptides in CFP samples containing the indicated species, where positive and negative results are indicated by red and blue boxes, respectively. All three Mtb peptides were detected in all but one Mtb-positive specimens, except one sample collected from a child who developed a rare, disseminated infection (BCGosis) after vaccination with M. bovis BCG, an attenuated strain that does not express CFP-10 or ESAT-6.

Automated MGIT systems need high bacterial biomass (10⁵ CFU/ml) to detect mycobacterial growth, and thus can require several weeks to detect a positive result and additional time for subcultures used for species identification. Since LC-MS/MS was found to robustly distinguish MGIT cultures at the first sign of culture positivity, whether this approach could also identify specific bacteria in MGIT cultures before mycobacterial growth was detectable was evaluated. CFP samples collected at successive intervals (day 7, 12, 15 and 21) from parallel MGIT samples generated from an Mtb isolate that was growth-positive at day 28, were analyzed using a modified PRM assay. This analysis employed all nine Mtb unique peptides that were reproducibly detected by PRM in 14 Mtb-positive MGIT cultures, see FIG. 10C.

DDA results for these serial MGIT culture samples detected 3 target peptides at day 15 and 7 target peptides at day 21, see FIG. 8D, all of which derived from either CFP-2 or ESAT-6, although the ESAT-6 peptides demonstrated the most consistent increase over time. PRM results for these samples detected 4 target peptides at day 7 and 6 target peptides at day 12, all of which also derived from CFP-2 and ESAT-6. Both the DDA and PRM results indicated that the number and relative expression of detected peptides progressively increased with time, although peptides derived from the same protein exhibited substantial signal intensity differences and not all of these consistently increased with culture time.

Two CFP-10 peptides detected in all of the MGIT growth-positive samples were not detected in any of these early growth samples by either method, indicating that different peptide markers may be required for early diagnosis in MGIT samples assessed prior to the first sign of mycobacteria growth. However, DDA and PRM data both permit more rapid identifications than obtained from Biotyper or WGS analyses of subcultures of growth positive MGIT samples used to confirm the identity of a mycobacterial species or its complex.

### Example Step 5—Results of Experiment

The identification of mycobacteria species and subspecies is frequently a complex and slow process that requires the use of multiple diagnostic protocols. The results, however, indicate that integration of a streamlined LC-MS/MS method with an automated PEP-TORCH algorithm analysis approach permits rapid and precise identification of mycobacteria at species, subspecies, or strain resolution through the detection of specific peptides and peptide combinations, including discrimination of subspecies that exhibit distinct drug resistance profiles. Rapid and precise diagnoses provided by PEP-TORCH thus have significant potential to improve patient outcomes, by facilitating early and accurate diagnosis to permit more rapid and effective treatment interventions.

Rapid and accurate subspecies and strain identifications can be important for epidemiology and appropriate treatment initiation, as some subspecies and strains have different inherent drug resistance or host interactions. Biotyper analyses have limited ability to distinguish drug sensitive and resistant subspecies of Mab and closely related species such as Min and M. chimaera, since they reply on detecting specific spectral patterns rather than specific protein or peptides. Their spectral libraries also may lack sufficient coverage to identify less common species or variants of more common species, problems that are less likely to affect LC-MS/MS that can discriminate samples based on specific peptide differences. Current clinical diagnostics can identify Mtb at a member of MTB complex, unlike PEP-TORCH results, which distinguished BCGosis from zoonotic TB caused by M. bovis and both from infections caused by other MTB complex species and strains. This is noteworthy since M. bovis exhibits inherent pyrazinamide resistance to affect treatment decisions.

In contrast to previous studies in using manual analysis to analyze limited peptide markers to identify species using a customized database, the pipeline utilized a universal mycobacterial database that could resolve all tested clinical NTM isolates, automated the search for peptides that could distinguish between different taxa levels, and provided scores that could be used to assist clinicians in diagnostic and therapeutic intervention decisions. In addition, PEP-TORCH has several advantages over mycobacterian identification methods since it can rapidly identify mycobacteria isolates to the subspecies and strain level with a single LC-MS/MS analysis. It does so by comparing detected biomarker peptides against protein database data to detect even single position differences in the peptide sequence of conserved proteins. This is a advantage over methods that use MS spectra data for identifications, since spectra differences, but not peptide identifications, can be affected by culture conditions. In this study, it is demonstrated that PEP-TORCH PWₛₚ and PW_{sub} scores can accurately identify species and subspecies in supernatants of early-growth clinical cultures, including co-infections. PEP-TORCH could also enhance identification of Mab subspecies that have different drug susceptibility. Currently the detection of drug resistant bacteria depends on drug susceptibility testing which involves culturing the pathogen and exposing to various antibiotics to detect susceptibility that could take several days to weeks. PEP-TORCH on the other hand, has the potential to rapidly identify subspecies and strains of pathogens, including those indicating drug resistance, without the need for protracted susceptibility testing. This expedited identification process can enable healthcare providers to make more informed and timely treatment decisions, leading to improved patient outcomes, especially in cases where prompt intervention is critical, such as in neonatal or immunocompromised patient populations.

It is important to note that PEP-TORCH's identifications of co-infection, hybrid infections, subspecies, and strains were consistent with clinical diagnoses obtained through various other clinical methods. However, a limitation of this study was the unavailability of WGS data for all specimens, which restricted the capacity to validate PEP-TORCH results concerning subspecies identification and co-infection. For instance, in the case of sample MKA3, both PEP-TORCH and Accuprobe concurred on its identification as Mka, but PEP-TORCH also generated a low Mtb score that could not be cross-checked with WGS data. The validation cohort, consisting of a total of 73 clinical samples, may not be particularly large in scale, but it retains its significance as a valuable resource. Significantly, it contributes to the comprehension of a substantial portion of the annual NTM cases in the United States, estimated at 4.7 cases per 100,000 person-years. This is a pathogen deserving of increased research attention, as its incidence is rising globally, and the development of diagnostic technologies for it lags behind. It is hoped that this study not only provides a novel detection platform but also raises awareness for further research targeting this pathogen.

In summary, this study highlights the ability of peptidomics data for rapid and specific identification of mycobacterial samples, particularly among closely related subspecies and strains. This should enhance the accuracy of species identifications within the mycobacterium superfamily to benefit clinical diagnosis and epidemiological studies. Further, the automated algorithm-based pipeline it uses can be employed as a universal means to analyze complex MS data from clinical isolates, as long as these isolates are represented in the MS databases used to evaluate the specificity of the detected peptides.

### Experimental Methods

### Sample Collection and Clinical Analyses

This study was reviewed and approved by the Tulane University Institutional Review Board (IRB) prior to study initiation. MGIT CFP samples analyzed in this study were obtained from clinical isolates of patients enrolled in IRB-approved protocols at the National Institutes of Health Clinical Center. All clinical isolates were inoculated into Bactec 960 MGIT tubes Remel; Lenexa, KS) containing Middlebrook 7H11 and PANTA growth supplement. Aliquots of growth-positive MGIT cultures were stained with auramine-rhodamine and gram stain to detect the presence of acid-fast bacilli (AFB) and/or contaminants and directly analyzed by WGS or sub-cultured on solid media and analyzed by Biotyper for species identification. Aliquots used for AFB were decontaminated using N-acetyl-L-cysteine/sodium hydroxide, centrifuged at for 15 min at 3,000×g, and the resulting pellets were suspended in 0.8 ml of sterile phosphate-buffered saline (PBS) and stained with auramine-rhodamine (Becton Dickinson, Sparks, MD) prior to analysis. Mycobacteria present in subcultures of growth-positive MGIT tubes were either directly identified by secA gene by whole genome sequencing and/or by MGIT subculture on solid media coupled with a subsequent Biotyper analysis. Species level differentiation within the MTB complex was determined by a multiplex PCR. MGIT CFP samples used for proteomics analysis were generated by passing 3 mL of all growth-positive MGIT cultures though 0.22 μm filters and storing the resulting filtrates at −80° C. until use.

### CFP Sample Fractionation

CFP samples were rapidly thawed to room temperature and aliquoted for subsequent analyses. One 100 μl aliquot of each sample was supplemented with an equal volume of acetonitrile and centrifuged at 16000 g for 25 min at 15° C. Supernatants were collected and precipitated overnight with 4 times volume of acetone overnight then centrifuged at 10000 g for 10 min at 4° C. Resulting pellets were re-dissolved in 50 mM ammonium bicarbonate. Protein concentrations were determined using a BCA protein assay reagent kit (Thermo Scientific, Rockford, Illinois, USA), SDS-PAGE gels (4-20% Mini-PROTEAN TGX Precast Protein Gels, BioRad, USA) were loaded with 10 μg aliquots of the supernatant and resuspended pellet material of each sample, subjected to electrophoresis, stained with Coomassie blue, imaged with a BioRad ChemiDoc Imaging System, and these images were processed and analyzed with ImageLab software (BioRad).

### Trypsin Digestion

A 50 μg aliquot of the acetonitrile-fractionation supernatant of each CFP sample was then reduced in 20 mM dithiothreitol for 10 min at 91° C. with mixing and then sonicated for 5 min. These samples were then adjusted to 25 mM iodoacetamide and alkylated for 20 min in the dark, before overnight digestion overnight at 37° C. using 1 μg of sequencing grade trypsin (Promega, USA). Resulting peptide samples were then acidified addition of trifluoroacetic acid (TFA) to a 0.1% final concentration. Styrenedivinylbenzene reverse phase sulfonate (SDB-RPS) stage tips used to process 20 μg aliquots of these samples were then prepared by packing SDB-RPS discs into 200 μl pipette tips with an 18-gauge syringe needle. Each stage tip was wetted by the addition of 50 μl of 100% acetonitrile and then centrifuged at 1000 g for 1 min, then equilibrated with 50 μl of 30% methanol/1% TFA and centrifuged at 1000 g for 3 min. Equilibrated stage tip were then loaded with 20 μg tryptic peptide aliquots and washed with 100 μl of a 99% propanol/0.1% TFA solution using a 1000 g for 2 min centrifugation step, after which peptides were eluted with 60 μl of elution buffer (80% acetonitrile, 1% ammonium hydroxide), dried, and reconstituted in 2% acetonitrile and 0.1% formic acid and subjected to DDA or PRM mode LC-MS/MS analysis for data acquisition.

### LC-MS/MS Analysis

For the DDA analyses, peptide samples were analyzed using an Ultimate 3000 nanoLC system (Thermo Fisher Scientific) coupled via a nano-electrospray ion source (Thermo Fisher Scientific) to a QExactive HFX Orbitrap (Thermo Fisher Scientific). Samples were loaded onto a 100 μm I.D.×2.5 cm, C18 trap column and a PepMap RSLC C18 (2 μm ID, 75 μm×25 cm) analytical column in buffer A (0.1% formic acid in water), Fractions were eluted with a linear gradient of 5% to 23% buffer B (80% acetonitrile in 0.1% formic acid) over 100 min. Following linear separation, the column was adjusted to 40% buffer B over 16 min and finally stepped to 98% buffer B for 4 min, then re-equilibration to 5% buffer B prior to the next injection. After adjusting each fraction to an estimated 0.5 to 1.0 μg on column, fractions were measured in a top-12 configuration with 20 s dynamic exclusion. Precursor spectra were collected from 300 to 1650 m/z at 60,000 resolutions (automated gain control (AGC) target of 3e6, max IT of 80 ms). MS/MS data were collected on +2H to +5H precursors achieving a minimum AGC of 2e3. MS/MS scans were collected at 15,000 resolutions (AGC target of 1e5, max IT of 120 ms) with an isolation width of 1.4 m/z with a normalized collision energy (NCE) of 27.

For the PRM analyses, samples were loaded onto a PepMap RSLC C18 column (3 μm ID, 75 μm×15 cm) at a 300 nl/min flow rate in Buffer A. Samples were eluted with a linear gradient of 5% to 28% buffer B over 46 min, and then the column was adjusted to 40% buffer B over 8 min, stepped to 98% buffer B for 6 min, and then re-equilibrated to 5% buffer B prior to the next sample injection. The MS1 had a resolution of 30000 and mass range between 150 and 2000 m/z; The maximum ion injection time was 200 ms with an AGC target of 3e6; The MS/MS had a resolution of 15000 and a maximum injection time of 100 ms with an AGC target of 2e5; the isolation window was 1.2 m/z. The NCE was set at 28 to 32 depending on the length of the target peptide sequence. All the peptide information targeted in the study is presented in the Supplementary Tables 21-25.

### Peptide Data Analysis

Raw MS data sets were de novo sequenced using PEAKS 10.5 software (Bioinformatics Solutions Inc., Waterloo, Canada) and the resulting peptide sequences were searched against the mycobacterium protein database (Uniprot taxon ID: 1762). The parameter of PEAKS are as follows: precursor tolerance is 20 ppm, fragment tolerance is 0.02 Da, three missed tryptic cleavages was allowed, dynamic modification is oxidation of Met. The PRM data were imported into Skyline for analysis.

### Taxonomy Analysis

High confidence peptides (−log₁₀ p>35) were searched against Unipept (https://unipept.ugent.be/) API code with the developed automated pipeline in R Studio to provide species identification. This threshold was decreased to −log₁₀ p>20 for peptides used for subspecies identification.

Peptide sets all samples were run through a PEP-TORCH algorithm built in the R Studio platform. Peptides were analyzed with Unipept API code to generate peptide-taxon matrixes that assigned unique peptides and specific taxa to the x- and y-axes of these matrixes. Each matrix entry was then screened to identify peptides that belonged to non-mycobacteria species or mycobacteria that did not derive from a clinical sample, which were then eliminated from the matrix. The PEP-TORCH algorithm then searched these reduced peptide-taxon matrixes to identify peptides that identified specific mycobacteria species and subspecies when they were used as single- or multi-peptide biomarkers in the form of pseudocode:

Box 1: Pseudocode used in PEP-TORCH

```
PEP-TORCH comprised two primary sections:
Identification Section: Filtering the unique peptides or combinations which were utilized as taxon
identification exclusively.
P - peptide set input in the algorithm
r - Number of peptides in the set
S - Taxon set
U - unique identified taxon
C - Combination event
C (1, r) - Single unique peptide candidate in r peptide set
C (2, r) - 2 - peptide combinations in r peptide set
C (3, r) - 3 - peptide combination in r peptide set
i, j, k, l, m, n - Peptide order
Sᵢ, Sⱼ, Sₖ, Sₗ, Sₘ, Sₙ - Taxa - peptide set; (S) taxa mapping to peptides (l, j, k)
(Sj [symbol]   Sk) - Intersection in 2 taxon-peptide set; if any species is common on 2-peptide- taxon set
(Sₗ [symbol]   Sₘ [symbol]   Sₙ) - Intersection in 3 taxon-peptide set; if any species is common on 3-peptide- taxon set
Input:
  - Total r peptides (P) identified from MS raw data; P = {p₁, p₂, ...,pᵣ}
  - Each peptide S = {s₁, s₂, ..., s_{T}};
Output: Unique identified taxon set (U)
U ← 0, Initialize the unique identified taxon
For 1 to C (1, r), ith peptide
If Element numbers of Si = 1, then
    U ← Si
    Remove the ith peptides from P
    For 1 to C (2, r-i), jth and kth peptide
    If element numbers of (Sj [symbol]   Sk) = 1, then
      U ← Sj [symbol]   Sk
      Remove the jth and kth peptides from the P
        For 1 to C (1, r-i-j-k), lth, mth, and nth peptides
        If element numbers of (Sl [symbol]   Sm [symbol]   Sn) = 1, then
          U ← Sl [symbol]   Sm [symbol]   Sn
Return U
Scoring Section: As the pipeline achieves final peptide-taxon matrixes for the shortlisted species and
subspecies based on their unique peptide combinations, the following system helped with analyzing the
PWₛₚ and PW_{sub} scores:
Input:
  - n x 3 unique peptide count matrix (T) for n identified taxons
  - p total counts of identified peptides
Output: Score of the identified taxons (S)
S ← 0, Initialize the score of the identified taxons
For 1 to n, the ith taxon:
    Total Combination Event (TCEi) = C(Tᵢ₁, p) + C(Tᵢ₁+Tᵢ₂, p) + C(Tᵢ₁+Tᵢ₂+Tᵢ₃, p)
    Si = TCEi / (TCE1 + TCE2 + ... + TCEn)
    S ← Si
Return S
```

What is claimed is:

1. A method for diagnosing a mycobacterial infection in a subject, the method comprising:
performing acetonitrile fractionation of a culture filtrate protein (CFP) sample obtained from the subject and retaining the supernatant fraction, wherein the retained supernatant fraction is selectively depleted in high molecular weight (HMW) proteins;
performing an enzymatic digest of the supernatant fraction to produce a digested peptide sample;
performing liquid chromatography tandem mass spectrometry (LC-MS/MS) and bottom-up proteomic analysis of the digested peptide sample to identify a plurality of sample peptides;
analyzing the plurality of sample peptides with a classifier algorithm, wherein the classifier algorithm is configured to cross reference each of the plurality of sample peptides with a mycobacterium peptide database to determine which of the plurality of sample peptides, individually or in combination, are specific to a mycobacterium species or subspecies in the database; and generating a PW species identification score (PWₛₚ) by dividing the number of single and multi-peptide combinations that identify a specific mycobacterium species by the total number of peptide combinations specific for any mycobacteria, wherein a higher (PWₛₚ) for a given species of mycobacterium indicates a higher likelihood the subject has an infection of that species.

2. The method of claim 1, wherein the classifier algorithm further comprises a first section, wherein the first section is configured to analyze and cross reference each of the plurality of sample peptides to a database and identify all species of organisms for which each of the plurality of sample peptides is associated.

3. The method of claim 2, wherein the classifier algorithm further comprises an identification section, wherein the identification section is configured analyze the output for the first section and eliminate all peptides not associated with a mycobacteria species and to output a refined plurality of sample peptides.

4. The method of claim 3, wherein the classifier algorithm further comprises a scoring section, wherein the scoring section analyzes the output of the identification section and eliminates all peptides that are not specific to a single mycobacteria species, either alone or combined with a second or third sample peptide and outputting a plurality of species-specific sample peptides.

5. The method of claim 4, wherein the classifier algorithm is further configured to compile the output of the scoring section and calculate the PWₛₚ score by dividing the number of species specific sample peptides (output of the scoring section) by the number of peptides in the refined plurality of sample peptides (output of the identification section).

6. The method of claim 5, wherein the classifier algorithm is further configured to repeat the steps of claims 4 and 5 for mycobacteria subspecies in order to generate a PW subspecies score (PW_{subsp}).

7. The method of claim 1, wherein a PWₛₚ score for a given species above a predetermined threshold indicates positive diagnosis of infection for the species.

8. The method of claim 7, wherein when two or more species have PWₛₚ score above the predetermined threshold, the subject is diagnosed with a co-infection

9. The method of claim 1, wherein the supernatant fraction is enriched in mycobacteria specific proteins.

10. The method of claim 1, wherein the supernatant fraction is comprised of proteins of about 60 kDa or less.

11. The method of claim 1, wherein the enzymatic digest is a trypsin digest.

12. The method of claim 1, wherein the CFP samples are from early-growth mycobacterial growth indicator tube (MGIT) cultures.

13. The method of claim 12, wherein CFP samples are collected and processed at the first sign of microbial growth.

14. A method for diagnosing a mycobacterial infection in a subject, the method comprising:
performing LC-MS/MS and bottom-up proteomic analysis on an enzymatically digested HMW depleted CFP sample from the subject and identify a plurality of sample peptides;
analyzing the plurality of sample peptides with a classifier algorithm, wherein the classifier algorithm comprises:
a first section, wherein the first section is configured to analyze and cross reference each of the plurality of sample peptides to a database and identify all species of organisms for which each of the plurality of sample peptides is associated;
an identification section, wherein the identification section is configured analyze the output for the first section and eliminate all peptides not associated with a mycobacteria species and to output a refined plurality of sample peptides;
a scoring section, wherein the scoring section analyzes the output of the identification section and eliminates all peptides that are not specific to a single mycobacteria species, either alone or combined with a second or third sample peptide and outputting a plurality of species specific sample peptides; and
wherein the classifier algorithm is further configured to compile the output of the scoring section and calculate the PWₛₚ score by dividing the number of species-specific sample peptides (output of the scoring section) by the number of peptides in the refined plurality of sample peptides (output of the identification section).

15. The method of claim 14, further comprising analyzing the plurality of peptides with classifier algorithm to generate a mycobacteria subspecies in order to generate a PW subspecies score (PW_{subsp}).

16. A system for diagnosing a mycobacterial infection in a subject comprising:
a module, configured to perform acetonitrile fractionation of a culture filtrate protein (CFP) sample obtained from the subject and retaining the supernatant fraction, wherein the retained supernatant fraction is selectively depleted in high molecular weight (HMW) proteins and to perform an enzymatic digest of the supernatant fraction to produce a digested peptide sample;
a module configured to perform liquid chromatography tandem mass spectrometry (LC-MS/MS) and bottom-up proteomic analysis of the digested peptide sample to identify a plurality of sample peptides;
one or more processors configured to: analyze the plurality of sample peptides with a classifier algorithm, wherein the classifier algorithm is configured to cross reference each of the plurality of sample peptides with a mycobacterium peptide database to determine which of the plurality of sample peptides, individually or in combination, are specific to a mycobacterium species or subspecies in the database; and
generate a PW species identification score (PWsp) by dividing the number of single and multi-peptide combinations that identify a specific mycobacterium species by the total number of peptide combinations specific for any mycobacteria, wherein a higher (PWsp) for a given species of mycobacterium indicates a higher likelihood the subject has an infection of that species.

17. The system of claim 16, wherein the classifier algorithm further comprises a first section, wherein the first section is configured to analyze and cross reference each of the plurality of sample peptides to a database and identify all species of organisms for which each of the plurality of sample peptides is associated.

18. The system of claim 17, wherein the classifier algorithm further comprises an identification section, wherein the identification section is configured analyze the output for the first section and eliminate all peptides not associated with a mycobacteria species and to output a refined plurality of sample peptides.

19. The system of claim 18, wherein the classifier algorithm further comprises a scoring section, wherein the scoring section analyzes the output of the identification section and eliminates all peptides that are not specific to a single mycobacteria species, either alone or combined with a second or third sample peptide and outputting a plurality of species-specific sample peptides.

20. The system of claim 19, wherein the classifier algorithm is further configured to compile the output of the scoring section and calculate the PWsp score by dividing the number of species-specific sample peptides (output of scoring section) by the number of peptides in the refined plurality of sample peptides (output of the identification section).
